(19) United States
(12) Patent Application Publication
(10) Pub. No.: US 2010/0298715 A1
(43) Pub. Date: Nov. 25, 2010

(54) METHOD AND MEASURING INSTRUMENT FOR COLLECTING SPECTROMETRIC TEST SIGNALS FROM LIVING TISSUE

(76) Inventors: Holger Jungmann, Gelsenkirchen (DE); Michael Schietzel, Herdecke (DE)

Correspondence Address: KF ROSS PC, 5683 RIVERDALE AVENUE, SUITE 203 BOX 900, BRONX, NY, 10471-0900, US

(21) Appl. No.: 12/680,047

(22) PCT Filed: Sep. 26, 2008
(86) PCT No.: PCT/EP08/08226

§ 371 Date: Aug. 11, 2010

(30) Foreign Application Priority Data
Sep. 26, 2007 (DE) 102007046257.5

(51) Int. Cl. A61B 6/00

(52) U.S. Cl. 600/476

(57) ABSTRACT

The invention relates to a method and a measuring instrument for collecting spectrometric test signals from living tissue. The aim of the invention is to create solutions which make it possible to generate, in the course of a spectrometric measurement, test results that supply more comprehensive data than previously known recording attempts. Said aim is achieved by a method for generating spectrometric test signals, in which light (L) is injected into a living tissue area (G) to be examined, reflected light (R) emerging from the tissue area to be examined is fed to a spectrometer device, and test signals representing the intensity of the reflected light by associating the same with the wavelength are generated by means of the spectrometer device. The measurement is taken such that the measurement process lasts a certain period of time (T) during which data is generated that represents the time course of the intensity of the resolved wavelengths, thus advantageously making it possible to generate, during the spectrometric measurement, signals which allow specific substances, e.g. the blood components cholesterol and sugar, to be associated with specific zones of the examined tissue area.

The invention relates to a method and a measuring instrument for collecting spectrometric test signals from living tissue.

Measurement methods are known, in which an analysis of vital tissue is done by applying a mobile spectrometer to a corresponding tissue area, and by this movable spectrometer the spectrum of reflected light emerging from the tissue is recorded. By means of the spectrum recorded in this way various substances present in the examined tissue area can be detected.

The object of the invention is to create solutions by which by means of a spectrometric measurement test results can be generated that supply more comprehensive data than the aforementioned former recording attempts.

This object is attained according to the invention by a method for generating spectrometric measuring signals in which method:

light is coupled into a living tissue area to be examined,

reflected light, that emerges as such from the tissue area to be examined, is led to a spectrometer device, and

by the spectrometer device test signals are generated, that represent as such the intensity of the reflected light associated to wavelength,

wherein the measurement is done in such a way that it extends over a lapse (T), and that for this lapse (T) data are generated which illustrate the time history of the intensity of the dissolved wavelengths.

It is thus advantageously possible during the spectrometric measurement to generate signals that allow as such an association of certain substances with specific zones of the examined tissue area.

Especially on the basis of the concept according to the invention it is possible to ascertain which of the substances emerging in the recorded spectra are subject to a vasomotion or other effects caused by vital processes. These substances can be associated to a subsystem by means of the kinematic profile recorded by the sequences of the spectra. Especially it is possible by the concept according to the invention to ascertain which substances are integrated into the bloodstream and which substances are integrated substantially statically into the tissue systems surrounding the bloodstream.

According to a particularly preferred embodiment of the invention the data indicative for the time course of the spectra are recorded by associating the spectrometrically resolved wavelengths to the data on the time course of the intensity. This storage can take place especially in that for recording the spectrum a data field is set up that contains data on every dissolved wavelength value which as such describe the time course of the intensity, especially the intensity dynamics. This data for intensity dynamics can be deposited especially as members of a series, especially FFT parameters.

Based on the dynamic characteristics recorded in this way an association of the single spectra contained in the summation spectrum as well as of the substances characterized by that with certain tissue, capillary, or fluid systems can be done.

As already indicated it is possible, based on the dynamic characteristics generated according to the invention, to ascertain if there is a substance in the bloodstream.

According to a further aspect of the invention it is also possible, using the dynamic characteristics ascertained according to the invention and based on the temporal changes of the summation spectra, to undertake a calculation of substance concentrations. These substance concentrations can be calculated especially based on an approach that takes into account that the total concentration of a substance detected over the total reflected light spectrum results from the partial concentrations of this substance in the single tissue systems, especially the tissue, capillary, or fluid systems.

The dynamic characteristics generated according to the invention for the temporal change of the reflected light spectrum can be used, according to a further aspect of the present invention, also as a basis for an evaluation method, in which by this evaluation method evaluation results are generated that as such describe or typify the physiological state of a person.

Further particulars and characteristics of the invention result from the following description in association with the drawing. The figures show:

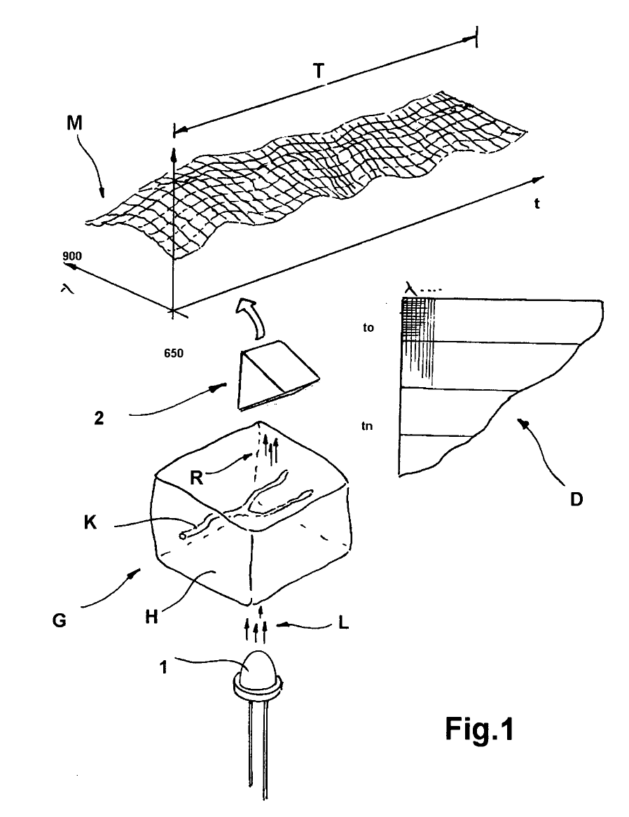

FIG. 1 a schematic representation to illustrate the approach according to the invention for generating a sequence of spectra and the use of the detected dynamic spectrum changes caused by vital processes for associating substances to certain tissue or capillary systems.

The representation according to FIG. 1 as such visualizes the method according to the invention for generating spectrometric test signals.

According to this process, by a light source 1 here configured as a diode, light L is coupled into a tissue area to be examined G. The light R emerging from this tissue area G is is conducted to a spectrometer device 2 indicated here only by way of example as a prism. By this spectrometer device 2 the light emerging from the tissue area to be examined is split into its spectral components. By this spectral splitting test signals M are generated that as such represent the intensity I (alternatively optical density OD) of the reflected light R associated to the wavelength. These test signals are continuously stored digitally.

The method according to the invention is characterized by the fact that the measurement is done in such a way that it extends over a lapse T and that for this lapse T data are generated which illustrate the time course of the intensity of the resolved wavelengths. In the measuring example indicated here the reflected light R conducted to the spectrometer device 2 comprises light from various areas of the tissue area G. Especially this light contains fractions which are caused for example by substances that flow through capillaries K of the tissue area G. Moreover the reflected light R also contains spectral components of substances that originate from areas H of the tissue area in which no particular dynamic changes of substance presences occur. The intensity of the spectra caused by substances that as such flow through the capillaries K, changes in the example shown here in accordance with a pulse pattern caused by vasomotion. This pulse pattern in the embodiment shown here is visible especially for certain wave spectral components in the range of 650 to 900 nanometers in the characteristic diagram recognizable here.

The concept according to the invention consists in is recognizing inside a summation spectrum the spectral contributions of those substances whose presence alternates by vital mechanical effects. In this way it is possible to isolate the spectrum of a pulsating fluid in a turbid medium.

In case of live tissue, especially perfused skin, the temperature of the static tissue corresponds substantially to the temperature of the pulsating fluid.

If the body is completely permeable, and if the fluid absorbs characteristically (like for example blood), then the pulsating current can be measured with the help of spectroscopic methods by absorption changes. Preferably the recording of the time history of the intensity is done with a resolution that supports evaluation-relevant vital kinematic effects for each interval with at least five measuring points.

If one measures now at various wavelengths, one obtains depending on the different absorption at different wavelengths, different pulse amplitudes. If one associates the amplitude differences to the wavelengths, one obtains a spectrum of the pulsating fluid. This difference spectrum is not disturbed by the ambient substance. If the spectrum of the fluid is known, mixed substances can be detected based on the absorption change compared to the known spectrum.

On the basis of the concept according to the invention it is especially possible to reliably recognize blood components, like cholesterol and sugar, optically.

1. A method for generating spectrometric test signals in which:
light is coupled into a living tissue area to be examined,
reflected light, that emerges as such from the tissue area to be examined, is led to a spectrometer device, and
by the spectrometer device test signals are generated, that as such represent the intensity of the reflected light associated to the wavelength,
wherein the measurement is done in such a way that it extends over a lapse, and that for this lapse data are generated which describe the time course of the intensity of the resolved wavelengths.

2. The process according to claim 1, wherein within the lapse a plurality of spectra are recorded.

3. The process according to claim 1 wherein the data indicative for the time course of the intensity of the resolved wavelengths are stored with association to the wavelengths.

4. The process according to claim 3, wherein for recording the spectrum a data field is set up, that contains data on every dissolved wavelength value, which as such describe the intensity curve or the intensity dynamics.

5. The process according to claim 1, wherein the data for intensity dynamics are deposited as FFT parameters.

6. The process according to claim 1 wherein by means of dynamic characteristics an association of spectra with tissue, capillary, and/or fluid systems is done.

7. The process according to claim 6, wherein based on the dynamic characteristics it is ascertained if there is a substance in the bloodstream.

8. The process according to claim 1 wherein a calculation of substance concentrations is done using the dynamic characteristics.

9. The process according to claim 1 wherein a calculation of the concentrations of the substances in the respective tissue, capillary, and/or fluid system is done using the dynamic characteristics.

10. The process according to claim 1 wherein the dynamic characteristics are used as a basis of an evaluation method, in which by means of this evaluation method evaluation results are generated that as such describe the physiological state of a person.

11. A process for the generation of a signal indicative for the vasomotoric state of a person in which light is coupled into a tissue area, the light emerging from the tissue area is spectrally split, and by means of the time course of the intensity of certain wavelengths, an evaluation result typifying the vasomotoric state of the person is generated.

12. A mobile spectrometer with a storage device and an evaluation circuit, which spectrometer is configured in such a way that by it a measurement can be realized, in which:
light is coupled into a living tissue area to be examined,
reflected light that emerges as such from the tissue area to be examined, is led to a spectrometer device, and
by the spectrometer device test signals are generated, that represent as such the intensity of the reflected light associated to the wavelength,
wherein the measurement is done in such a way that it extends over a lapse, and that for this lapse data are generated which describe the time course of the intensity of the resolved wavelengths.
